SEQUENCE LISTING (9 sequences)

SEQ ID NO: 1 - DNA, 49 bp, Artificial
  Primer U1A P1
aattctaatacgactcactatagggagaggcccggcatgtggtgcataa

SEQ ID NO: 2 - DNA, 20 bp, Artificial
  Primer U1A P2
tgcgcctctttctgggtgtt

SEQ ID NO: 3 - DNA, 34 bp, Artificial
  Probe U1A MB
cgcatgctgtaaccacgcactctcctcgcatgcg

SEQ ID NO: 4 - DNA, 51 bp, Artificial
  Primer AC133 P1
aattctaatacgactcactatagggaagaacagggatgatgttgggtctca

SEQ ID NO: 5 - DNA, 26 bp, Artificial
  Primer AC133 P2
tttcaaggacttgcgaactctcttga

SEQ ID NO: 6 - DNA, 30 bp, Artificial
  Probe AC133 MB
cgatccaaggacaaggcgttcacaggatcg

SEQ ID NO: 7 - DNA, 61 bp, Artificial
  Primer EST032 P1
aattctaatacgactcactatagggagtagcccactcaagagctctctcctgttggtccct

SEQ ID NO: 8 - DNA, 35 bp, Artificial
  Primer EST032 P2
gcatctctgttcatgactgtgtgagctcctgtcct

SEQ ID NO: 9 - DNA, 32 bp, Artificial
  Probe EST032 MB
cgtacgaatgacgtgcccctgcgaatcgtacg

(19) United States
(12) Patent Application Publication
(10) Pub. No.: US 2009/0098563 A1
(43) Pub. Date: Apr. 16, 2009

(54) Diagnosis of (a risk of) disease and monitoring of therapy

(75) Inventors: Maarten Tjerk Penning, Utrecht (NL); Sebastiaan Johannes Jacobus van den Broek, Heerhugowaard (NL); Emile Eugene Voest, Soest (NL); Laurens Victor Beerepoot, Utrecht (NL); Niven Mehra, Utrecht (NL)

Correspondence Address: TRASK BRITT, P.O. BOX 2550, SALT LAKE CITY, UT, 84110, US

(73) Assignees: PrimaGen Holding B.V.; UMC Utrecht Holding B.V.

(21) Appl. No.: 12/284,203

(22) Filed: Sep. 19, 2008

Related U.S. Application Data
(63) Continuation of application No. 11/514,345, filed on Aug. 31, 2006, now abandoned.
Application No. 11/514,345 is a continuation of application No. PCT/NL2005/000155, filed on Mar. 2, 2005.
(60) Provisional application No. 60/549,450, filed on Mar. 2, 2004.

(30) Foreign Application Priority Data
Mar. 2, 2004 (EP) 05710924.1

(51) Int. Cl. C12Q 1/68; C07H 21/04

(52) U.S. Cl. 435/6; 536/24.31; 536/24.33

(57) ABSTRACT

Provided are methods for typing a sample of an individual suffering from, or at risk of suffering from, a disease and a method for monitoring treatment of an individual suffering from a disease comprising determining whether a sample from the individual comprises an expression product of AC133 in an amount that is indicative for the disease or for the treatment thereof. That amount may be quantified and compared with a reference value. In one aspect, the amount is compared with an amount of the expression product present in a sample that was obtained from the individual before treatment. Use of a nucleic acid molecule comprising at least part of a sequence of AC133, or an analogue thereof, for monitoring a treatment of an individual suffering from a disease is also provided, as well as a diagnostic kit comprising such nucleic acid molecule.

### CROSS-REFERENCE TO RELATED APPLICATIONS

This application is a continuation of co-pending U.S. patent application Ser. No. 11/514,345, filed Aug. 31, 2006, which application is a continuation of PCT International Patent Application No. PCT/NL2005/000155, filed on Mar. 2, 2005, designating the United States of America, and published, in English, as PCT International Publication No. WO 2005/083123 A1 on Sep. 9, 2005, which PCT application claims priority to European Patent Application Serial No. 04075686.8 filed on Mar. 2, 2004, and to U.S. Provisional Patent Application Ser. No. 60/549,450, also filed on Mar. 2, 2004, the contents of the entirety of each of which are hereby incorporated herein by this reference. This application also claims the benefit under 35 U.S.C. § 119(e) to U.S. Provisional Patent Application Ser. No. 60/549,450, also filed on Mar. 2, 2004.

### TECHNICAL FIELD

The invention relates to the field of medicine. The invention particularly relates to the fields of molecular biology and detection methods.

### BACKGROUND

Recent advances in the knowledge of molecular processes in an organism and techniques to study these processes have resulted in improved methods of typing and treating diseases. Research is being carried out in many fields in order to provide and/or improve methods for diagnosis and treatment of disease, as well as providing and/or improving methods for monitoring (side) effects of treatment.

Currently, treatment involving counteracting or enhancing angiogenesis is widely used for a broad spectrum of diseases. Angiogenesis (generation and/or maintenance of blood vessels) often plays an important role in recovery from disease. For instance, active growth of blood vessels is involved with regenerative treatment. Treatment of heart and coronary diseases aims at the generation of a new blood supply to affected organs by means of new blood vessels.

Angiogenesis is also involved with the onset and/or development of many different kinds of diseases, such as tumor growth. It is well established that the growth of tumors beyond 1 to 2 mm³ is dependent upon the formation of new blood vessels. On the one hand, blood vessels are required to carry nutrients to the site of the tumor, whereas on the other hand, waste material needs to be transported from the tumor.

Angiogenesis can be triggered by tumors by secretion of specific chemokines or cytokines. During angiogenesis, endothelial cells proliferate and become motile, moving towards the source of the angiogenic stimulus (e.g., the tumor), degrading the basement membrane and forming primitive vessels. At the same time, the cells increase their proliferative rate from near quiescent to approaching that of bone marrow. This new growth occurs in response to a number of vascular growth factors (J. Folkman, Nature Med. 1:27-31, 1995; Miller, Breast Cancer Res. Treat., 2002). In view of this dependency on angiogenesis, tumor treatment often involves anti-angiogenesis drugs.

Because of the common role of angiogenesis during the course of disease and treatment, current methods of diagnosing and staging of disease, and/or methods for monitoring the treatment of disease, are often focused on endothelial cells, since endothelial cells proliferate and become motile during angiogenesis. Mancuso et al. determined the number of circulating endothelial cells in cancer patients as compared to healthy controls. They reported an increased number of circulating endothelial cells in 76 cancer patients. Recently, it was shown that in cancer patients, circulating endothelial cells are increased during progressive disease, and that patients with stable disease had circulating endothelial cell numbers equivalent to healthy volunteers (L. Beerepoot, Ann. Oncol. 15:139-145, 2004).

### DISCLOSURE OF THE INVENTION

Provided are methods for determining whether an individual is suffering from or is at risk of suffering from a disease. Further provided are methods for determining whether a tumor is stable or progressive. Also provided are methods for monitoring treatment of an individual suffering from a disease.

Demonstrated is that the amount of an expression product of AC133 (SEQ ID NOS:4-6) in a sample from an individual is indicative for a disease or for the treatment thereof. It is, for instance, shown that the expression of AC133 in untreated cancer patients is significantly higher compared to healthy individuals. There is also shown in the examples that AC133 expression significantly drops when various tumor patients are treated, while the total number of circulating endothelial cells remains essentially the same during the same treatment. Hence, according to the invention, the amount of AC133 expression product is indicative for disease or for the treatment thereof.

Provided are methods for typing a sample of an individual suffering from, or at risk of suffering from, a disease comprising determining whether the sample from the individual comprises an expression product of AC133 in an amount that is indicative for the disease or for the treatment thereof. In one aspect, a method of the invention is used for the diagnosis of disease. People can be routinely tested with a method of the invention within certain time intervals.

Alternatively, people can be tested when clinical symptoms occur. An unusual amount of AC133 expression product in a sample of the individual, as compared to natural amounts in healthy individuals, is indicative for (a risk of) a certain degree of disease.

The invention furthermore provides a method for monitoring treatment of an individual suffering from a disease comprising determining whether a sample from the individual undergoing the treatment or having undergone the treatment, comprises an expression product of AC133 in an amount that is indicative for the treatment. As defined herein, typing a sample of an individual means determining whether the sample is indicative for disease or for the treatment thereof. Monitoring treatment of an individual means that therapeutic activity and/or possible side effects of the treatment are determined, preferably during a certain time interval. Therapeutic activity means the capability of at least in part treating a disease.

In certain embodiments the therapeutic activity comprises a therapeutic activity against a tumor-related disease and/or a blood vessel-related disease. A blood vessel-related disease is defined herein as a disease involving generation, maintenance and/or breakdown of blood vessels.

AC133, also called CD133, was first described in 1997. It was supposed to be a marker for human hematopoietic stem and progenitor cells (Yin, Blood, 1997; Miraglia, Blood, 1997). Most of the CD34+VEGF-R2+ endothelial cells express AC133. Mature endothelial cells do not express AC133.

Recently, a second isoform of AC133 with a 26 nucleotide deletion was described. This isoform, called AC133-2, is the isoform that is expressed on hematopoietic stem cells. The surface antigen that is recognized by anti-AC133 monoclonal antibodies in the art that are used for the isolation of hematopoietic stem cells recognize AC133-2 and not AC133-1 (Yu, J. Biol. Chem., 2002).

The length of the AC133 mRNA is 3794 nucleotides. By comparison of the mRNA sequence (GenBank accession: AF027208) to a sequence of 115812 nucleotides of genomic homo sapiens chromosome 4 (GenBank accession: NT_{—}006344), the total coding sequence of AC-133 turns out to comprise 27 exons.

An “individual” is defined herein as an animal having blood vessels. Preferably, the animal is a mammal, such as a human.

In a method of the invention, it is determined whether a sample comprises an expression product of AC133 in an amount that is indicative for disease or for treatment thereof. According to the invention, the amount of AC133 expression product is correlated to the status of an individual. A diseased individual has an altered AC133 expression as compared to a healthy individual. Moreover, treatment of a disease can be monitored by determining the amount of AC133 expression product, preferably at several time points. The amount of AC133 expression product is determined using any method known in the art. The art provides various methods for determining the amount of AC133 expression product.

In certain embodiments, the AC133 expression product comprises protein. The amount of protein is, for instance, determined using (capture) ELISA, Western blotting, a biosensor, etc.

In certain embodiments, the amount of protein is compared with a reference value, preferably the amount of protein present in a comparable sample of the same individual before the start of therapy. Alternatively, mean values of comparable samples of a healthy and/or diseased population are used as a reference.

The AC133 expression product may comprise RNA. The RNA may comprise mRNA because mRNA has a short half-life and the amount of mRNA, therefore, more accurately reflects the actual status of AC133 expression. In order to detect RNA, an amplification reaction, such as a NASBA amplification reaction, is often preferred. Specific amplification of a target nucleic acid sequence is achieved by adding two primer sequences to an amplification reaction mixture. The original amount of RNA can be determined in various ways. An amplified region is, for instance, detected at the end of an amplification reaction by probes that are specific for the amplified region.

Alternatively, an amplified region is detected during generation of the amplified nucleic acid in the amplification reaction.³ In the latter protocol, a signal of a label attached to a probe becomes detectable after the probe has hybridized to a complementary nucleic acid. Examples of such probes that enable real-time homogenous detection in amplification reactions are TaqMan³ and Molecular Beacon probes.^{4, 5}

Preferably, the amount of AC133 RNA is compared with a reference value, for instance, the amount of AC133 RNA present in a comparable sample of the same individual before the start of therapy. Alternatively, mean values of comparable samples of a healthy and diseased population are used as a reference.

By a “comparable sample” is meant the same kind of sample. Hence, if the amount of AC133 expression product in a blood sample is measured after start of therapy, a “comparable sample” means another blood sample, preferably taken before the start of therapy. Preferably, the sample obtained after start of therapy and the comparable sample are similarly processed. In one aspect of the invention, the same kind of nucleic acid amplification reaction is performed with both samples.

In certain embodiments, samples that are compared with each other, for instance, samples that are taken before and after treatment, contain essentially the same volumes (liquid samples) or sizes (tissue samples). Alternatively, the samples contain different volumes/sizes. In that case, the differences between the volumes/sizes of the samples are taken into account when comparing the amount of expression product. For instance, if a blood sample taken after start of therapy contains only half of the volume of a sample taken before treatment, the measured amount of AC133 in the sample taken after start of therapy should be multiplied by two in order to allow a direct comparison with the amount of AC133 expression product in the sample taken before treatment. Of course, it is also possible in that case to divide the amount of AC133 expression product present in the sample taken before treatment by two in order to directly compare it with the amount of AC133 expression product present in the sample taken after start of treatment.

A change in the amount of expression product of AC133 is indicative for whether a treatment is effective or not. In certain embodiments, this change in the amount of AC133 expression product is due to an altered expression by cells involved with the disease, for instance, by tumor cells and/or surrounding tissue. In certain embodiments, however, AC133 expression of other cells that are not directly involved with disease, such as cells in blood circulation, is determined.

In certain embodiments, the amount of AC133 expression product is reduced when a treatment is effective. In this case, it is determined whether a treatment is effective by determining whether the amount of AC133 expression product reduces over time. To enable more accurate comparisons with a reference value, the amount of AC133 expression product is preferably quantified. Known methods in the art are suitable for this purpose. Quantification of a target nucleic acid sequence is commonly accomplished by adding a competitor molecule, which is amplified using the same primers and which contains sequences that allow discrimination between competitor and target nucleic acid sequence.^{2, 6} The ratio between amplified competitor and target nucleic acid sequence is used to quantify the target nucleic acid sequence.

Detection of competitor or target nucleic acid sequence is, for instance, achieved at the end of the amplification reaction by probes that are specific for the amplified region of competitor or target nucleic acid sequence or during generation of the amplified nucleic acid in the amplification reaction. In the latter protocol, a signal of a label attached to a probe can become detectable after the probe has hybridized to a complementary target nucleic acid and when the target has exceeded a threshold level; the time or cycle number to positivity. In other methods for quantification, the time to positivity is used for quantification without addition of a competitor.⁷

Alternatively, the original amount of nucleic acid is established by determining the amplification rate of the nucleic acid during an amplification reaction, as outlined in PCT application PCT/NL03/00780 of the present applicant which is incorporated herein by reference. According to PCT/NL03/00780, a nucleic acid amplification rate is indicative for the amount of nucleic acid initially present in a sample before amplification.

In certain embodiments, the absolute amount of AC133 expression product is determined. In a preferred embodiment, however, the relative ratio of an AC133 expression product is determined in relation to another nucleic acid (for instance, DNA and/or RNA) or gene product thereof (derivable by transcription and/or translation, such as mRNA and/or a (poly)peptide) present in a sample obtained from the individual. In terms of the invention, by a “relative ratio” is meant the amount of the AC133 expression product in relation to the amount of the other nucleic acid and/or gene product thereof. The relative ratio can, for instance, be determined by (amongst other things) dividing the amount of the AC133 expression product by the amount of the other nucleic acid or gene product thereof, or vice versa. The amount of one or both products can also be divided by, or subtracted from, a reference value. Preferably, the other nucleic acid and/or gene product thereof comprises nuclear nucleic acid and/or gene product thereof. “Nuclear nucleic acid,” as defined herein, comprises chromosomal DNA and/or RNA transcribed therefrom. More preferably, the other nucleic acid and/or gene product thereof is stable and/or abundantly present in a cell, such as DNA or corresponding mRNA encoding components of small nuclear ribonucleoprotein (snRNP), and/or other essentially common nucleic acid or gene product thereof derived from chromosomal DNA.

In certain embodiments, the other nucleic acid or gene product thereof comprises U1A (SEQ ID NOS:1-3) or Beta-Actin RNA. When the number of AC133 mRNA copies is compared to the number of nuclear nucleic acid or gene product thereof, the amount of AC133 expression product is preferably expressed as copies per cell.

For instance, if the AC133 expression product comprises protein, a relative ratio between the AC133 protein and at least one other protein in the sample is preferably determined. The relative ratio is preferably compared with a reference value. The other protein is preferably abundantly present. For instance, the relative ratio between the AC133 protein and a (preferably abundantly present) housekeeping protein is determined. In another embodiment, a relative ratio between the amount of AC133 in a sample and the amount of total protein in a sample is determined.

If the AC133 expression product comprises RNA, preferably mRNA, a relative ratio between the AC133 RNA and at least one other nucleic acid in the sample is preferably determined. The relative ratio is preferably compared with a reference value. The other nucleic acid is preferably abundantly present.

In certain embodiments, the other nucleic acid comprises U1A. In another embodiment, a relative ratio between AC133 RNA, preferably mRNA, in a sample and the amount of total nucleic acid (be it total RNA, total DNA or total RNA+DNA) in a sample is determined.

In certain embodiments, the AC133 expression product and the other nucleic acid or gene product thereof both comprise nucleic acid. Minute amounts of target nucleic acid can be detected and quantified by using enzymatic amplification reactions, such as (RT)-PCR, NASBA, SDA, TMA, bDNA or Rolling Circle amplification.

In certain embodiments, both kinds of nucleic acid are amplified separately and the initial amounts are determined separately. Preferably, however, both nucleic acid sequences are amplified in the same assay (called herein a duplex amplification reaction) because in that case, double spreading in the result is avoided, as outlined in WO 02/46470 of the present applicant, incorporated herein by reference.

Generally, double spreading in a result is obtained due to varieties in conditions in different reaction mixtures. For instance, with amplification reactions, the temperature of the reaction mixture of nucleic acid 1 may be slightly higher than the temperature of the reaction mixture of nucleic acid 2. This may result in a higher yield of nucleic acid 1 and, hence, in a higher ratio of the amount of nucleic acid 1 versus nucleic acid 2 than would have been obtained if the temperature of reaction mixture 1 had been exactly the same as the temperature of reaction mixture 2. Because of the temperature difference in the reaction mixtures, the determined ratio is not exactly the same as the real ratio of the two nucleic acids present in the initial sample. Likewise, minute variations in other conditions like, for instance, the amount of enzyme added, can lead to variations in the determined amounts of nucleic acids 1 and 2. Thus, in separate amplification reactions, the measured amounts of nucleic acids 1 and 2 may vary independently from each other. Independent variations in the determined amounts may result in variation in a calculated ratio of the measured amounts. This is called “double spreading in the result.” Thus, by “double spreading” is meant herein at least one variation in an obtained result, due to a variety of at least one reaction condition in at least two reaction mixtures. For instance, the temperature and/or the total amount of volume may differ slightly between two reaction mixtures.

Double spreading is, for instance, prevented by determination of the ratio in the same assay. This means that a processing step and/or a measurement of the amounts of at least two nucleic acids and/or gene products thereof is performed in the same assay. In terms of the invention, an assay typically utilizes one reaction mixture. All components of an assay may be mixed randomly in the assay. The reaction mixture is preferably present in one reaction tube.

There are more methods to prevent double spreading in the result. For instance, a reaction vessel may be used that is divided in different parts by a (semi)permeable membrane. As long as at least one reaction condition varies dependently in the different parts, double spreading is avoided and the obtained result will be even more accurate.

In certain embodiments, AC133 RNA and a second nucleic acid are amplified in one assay. When both nucleic acid sequences are amplified in one assay, the same varieties in reaction conditions in the assay will influence the obtained amount of each sequence. For instance, the obtained amount of each sequence present in the assay will be influenced by the same temperature, the same overall volume, and so on. Detection of the two target sequences can be achieved by using two specific probes during the generation of amplified nucleic acids in an amplification reaction. Preferably, the two probes each have a different label allowing discrimination between the two probes and thereby between the two different target sequences.

Quantification is, for instance, achieved by relating the time to positivity as well as the slope of the relative fluorescence increase of both real-time amplification reactions. Preferably, a reference curve is created before quantification. The quantification of the nucleic acid is then performed by comparing the obtained value(s) with the reference curve. Thus, there is no need for an internal standard like, for instance, a competitor molecule. A method of relative quantification of two targets in one assay has an improved accuracy compared to quantification in two separate assays, and requires less handling time and reagents. Duplexing of two amplification reactions in the same tube gives an immediate indication of the ratio of the two targets. In certain embodiments, dividing one amount of nucleic acid by another is performed by dividing the intensity of the corresponding fluorescent label by another.

In certain embodiments, at least one sample from the individual is obtained before the treatment and at least one sample from the individual is obtained after initiation of the treatment. In certain embodiments, several samples from the individual are obtained at different time points after initiation of treatment. This enables monitoring the course of treatment during a prolonged period. It can, for instance, be determined whether the amount of AC133 expression product remains indicative for the disease or the treatment thereof. This is, for instance, useful for establishing appropriate treatment schedules, dosage and type on a patient-per-patient basis. Furthermore, it can be determined whether continuation of treatment at a given time point is appropriate. For instance, during tumor treatment, the amount of AC133 mRNA drops. If the amount of AC133 mRNA remains low as compared to the amount of AC133 mRNA in a sample obtained before treatment, it indicates that the treatment remains effective. If, however, the amount of AC133 initially drops but subsequently rises again, this indicates that the effectiveness of the therapy diminishes. In that case, the dosage of the medicament(s) is optionally increased. If the amount of AC133 mRNA lowers again after an increased dosage, it indicates that such higher dosage is more effective in counteracting disease. If, however, the amount of AC133 expression product does not fall and/or does not remain low, one may decide to stop the therapy. Another therapy, if available, is chosen, which is also monitored with a method of the invention.

In general, as long as measurements at different time points indicate that an AC133 expression product is altered as compared to the amount of AC133 expression product in a comparable sample taken before therapy, it indicates that the therapy is effective.

With a method of the invention, it is possible to determine whether a treatment is effective in an individual. This can be done while a treatment is given or shortly after the treatment or part thereof has ended. Thus, it is possible, for instance, to adjust the treatment schedule, dosages and type on a patient-per-patient basis. It is preferred that the sample is obtained within a month of initiation of treatment. More preferably, the sample is obtained within a week, and most preferably within two days of initiation of treatment because an early estimation of effectiveness of therapy allows for early adjustment of the treatment schedule, dosages and type. With a method of the invention, it is possible to evaluate treatment effectiveness almost immediately after initiation of the treatment, especially when the amount of AC133 mRNA is determined. A method of the invention thus allows easy, early monitoring of treatment, whereas current methods, such as analyzing biopsy samples and radiological analysis of tumor cells, require complicated, expensive and/or time-consuming procedures.

If the (mean and/or relative) amount of AC133 expression product in a sample of an individual is compared to a reference value (such as, for instance, the amount of AC133 expression product in a comparable sample of the same individual before the start of therapy, or a mean value of comparable samples of a healthy and/or diseased population), the difference between the reference and the (mean and/or relative) amount of AC133 expression product in the sample is preferably greater than or equal to the standard deviation of the reference. More preferably, the difference is greater than or equal to two times the standard deviation of the reference. Most preferably, the difference is greater than or equal to three times the standard deviation of the reference.

If a (PBMC) sample is used without further significant purification of cells, the amount of AC133 expression product in the sample is preferably at least two times higher or lower (depending on whether disease or treatment of disease is measured) than the reference value. More preferably, the amount of AC133 expression product in the sample is at least four times higher or lower than the reference value. Most preferably, the amount of AC133 expression product in the sample is at least ten times higher or lower than the reference value. Of course, the (absolute) difference between the amount of AC133 expression product in a sample of an individual and a reference value is dependent on the specific kind of sample used and/or on the relative ratio of the amounts of AC133 expression product and another expression product.

The difference in the (relative) amount of AC133 expression product in an effective and a non-effective treatment can be very large. In the extreme cases, the level of AC133 expression product ranges from detectable to not detectable. A zero-to-one relation can be used to design relatively simple test systems. A zero-to-one relation is, of course, dependent on the detection system used to detect AC133 expression product. Very sensitive expression detection systems will typically detect expression product where a less sensitive system detects no expression product. The most appropriate expression detection system may be designed to practice this aspect of the invention.

In certain embodiments, a method of the invention is provided wherein the disease comprises the presence of a tumor. According to the invention, the amount of AC133 expression product is altered in an individual suffering from, or at risk of suffering from, a tumor-related disease as compared to the amount of AC133 expression product in an individual who is not, or to a significantly lesser extent, suffering from, or at risk of suffering from, a tumor-related disease. Furthermore, a method of the invention is suitable for determining whether a tumor is progressive. A progressive tumor is defined herein as a tumor with a significantly growing tumor mass and/or a tumor that is involved in the development and/or presence of at least one metastasis and/or circulating tumor cells originating from the tumor. Contrary, the mass of a stable tumor is slowly, if at all, growing and a stable tumor is not or barely involved in development of metastases.

According to the invention, a (relative) amount of AC133 expression product is higher when a tumor is progressive as compared to a stable tumor. In certain embodiments, a method of the invention is, therefore, used for determining whether an individual is suffering from, or at risk of suffering from, a progressive tumor. This embodiment is particularly suitable if an individual is suffering from a type of tumor that is generally involved in a modest increase of AC133 expression product, as compared to other kinds of tumors.

According to the invention, various kinds of tumors are involved with different increases in AC133 expression product. This is, for instance, shown in FIG. 17. Breast cancer and colorectal cancer are involved in higher increases in AC133 expression product as compared to ovarian cancer, prostate cancer and renal cell carcinoma. Hence, especially when an individual appears to be suffering from a type of tumor that is involved in a relatively modest increase of AC133 expression product, a high amount of AC133 expression product indicates that the tumor is progressive.

In another preferred embodiment, a method of the invention is used for monitoring the status of a tumor over time. In this embodiment, the amount of AC133 expression product is determined in at least two samples taken from an individual at different time points. If the amount of AC133 expression product in an individual appears to be declining over time, it indicates that the tumor has become (more) stable and/or that regression has occurred. A declining amount of AC133 in an individual is, for instance, determined when the amount of AC133 expression product in a sample taken from the individual at a later time point comprises less AC133 expression product as compared to the same kind of sample taken from the individual at an earlier time point. If, however, the amount of AC133 expression product in an individual appears to be (suddenly) rising, it indicates that a tumor has become more progressive and/or that a cured patient experiences a cancer relapse.

In certain embodiments, a method of the invention is, therefore, used for monitoring the status of a tumor. In a preferred embodiment, a method of the invention is used for monitoring the status of a tumor during treatment in order to assess whether a treatment is effective in at least partly counteracting the tumor. If a tumor becomes less progressive, a treatment is effective. Moreover, a method of the invention is preferably used after tumor treatment, for instance, in order to monitor whether a stable tumor remains stable or becomes progressive and/or to monitor whether a (progressive) tumor evolves (for instance, whether a cured individual experiences a cancer relapse).

The invention thus provides a method for determining whether a treatment of an individual is effective in, at least in part, counteracting a progressive tumor, comprising determining whether a sample from the individual comprises a lower (relative) amount of AC133 expression product as compared to the same kind of sample of the individual taken at an earlier time point.

In a preferred embodiment, the (relative) amount of AC133 RNA, preferably mRNA, in a sample is determined. This is preferably performed using a nucleic acid molecule comprising at least part of a sequence of AC133 or an analogue thereof as a primer and/or probe. The length of the nucleic acid molecule or analogue is preferably at least four nucleotides. Preferably, the length is at least five nucleotides, more preferably at least six nucleotides, even more preferably at least seven nucleotides, yet more preferably at least eight nucleotides, even more preferably at least nine nucleotides, and most preferably at least ten nucleotides in order to enhance specificity of the primer and/or probe. The nucleic acid molecule or analogue is furthermore preferably shorter than 150 nucleotides in order to allow efficient binding. The nucleic acid molecule or analogue preferably comprises between about 15 and about 30 nucleotides. The length of the part of an AC133 sequence is preferably at least 50%, more preferably at least 60%, even more preferably at least 70%, yet more preferably at least 80%, even more preferably at least 90%, and most preferably at least 95% of the length of the nucleic acid molecule.

One embodiment thus provides a use of a nucleic acid molecule comprising at least part of an AC133 sequence or an analogue thereof for determining whether a tumor is progressive.

A use of a nucleic acid molecule comprising at least part of a sequence of AC133 or an analogue thereof for monitoring a treatment of an individual suffering from a disease comprising the presence of a progressive tumor is also herewith provided.

Alternatively, or additionally, it is possible to use another kind of molecule capable of specifically binding an AC133 expression product. Therefore, also provided is a use of a molecule able to specifically bind an AC133 expression product for determining whether a tumor is progressive.

Moreover, treatment of the tumor-related disease can be monitored with a method described herein. In certain embodiments, the tumor is mouth bottom carcinoma, adenoidcystic carcinoma, renal cell carcinoma, colon carcinoma, an esophagus tumor, mesothelioma, pancreas tumor, bladder tumor, adenocarcinoma of unknown primary (ACUP), prostate tumor, renal adenocarcinoma, head and neck cancer and/or malignant melanoma.

As is shown in the examples, the amount of AC133 mRNA is significantly lowered during treatment of these diseases. It is also shown in the examples that the expression of AC133 per 10,000 copies U1A DNA or Beta-Actin DNA in various untreated cancer patients is almost one log higher compared to healthy donors. The extent of increase in AC133 expression product is dependent on the kind of tumor.

Therefore, in a further preferred embodiment, the tumor comprises breast cancer, colorectal cancer, prostate cancer and/or ovarian cancer because these types of tumors are particularly associated with an increase of AC133 expression product, as is shown in FIG. 17. Most preferably, the tumor comprises breast cancer and/or colorectal cancer, since these tumors are involved in high increases of the amount of AC133 expression product.

In yet another embodiment, a method of the invention is provided wherein the disease is a blood vessel-related disease. As used herein, by a “blood vessel-related disease” is meant a disease that involves generation, maintenance and/or breakdown of blood vessels. Preferably, the disease comprises heart disease, high blood pressure, transient ischemic attacks and strokes, psoriasis, Crohn's disease, rheumatoid arthritis, endometriosis, atherosclerosis, obesity, diabetes, diabetic retinopathy, macular degeneration, Alzheimer's disease, Peutz-Jegher's syndrome, multiple sclerosis, systemic lupus erythematosus, Wegener's granulomatosis, vasculitis, sickle cell disease, thalassemia and/or angina.

A healing process can be followed with a method of the invention. For instance, recovery of damaged tissue involves an altered amount of AC133 expression product over time. Samples taken at different time points provide information about the amount of AC133 expression product that is generated during different time intervals. An altered amount of AC133 expression product found in samples during a period of time is, for instance, indicative for generation of tissue cells. An important application is treatment of heart and coronary disease. A method of the invention is suitable for monitoring the generation of new cardiac tissue.

In one aspect, a method is provided wherein the sample comprises a significant amount of non-endothelial cells. It has been shown by the present inventors that the number of circulating endothelial cells is not always indicative for the status of an individual, while the total amount of AC133 expression product is indicative for the status. Preferably, the sample is an essentially cell-free sample.

In a preferred embodiment, a sample of a method of the invention is a blood sample, although the location of, for instance, an angiogenic process can be a tumor or a part of the skin. A blood sample is preferred, amongst other things, because it is much easier to obtain and relatively large amounts are often available. A blood sample is also often easier to investigate, requiring less expensive and/or specific equipment.

Quite surprisingly, we have found that the expression of AC133 by hematopoietic cells, like peripheral blood mononuclear cells (PBMCs), is indicative for a process occurring somewhere else in an individual's body. For instance, the alteration in amount of an AC133 expression product in PBMCs is indicative for the presence of a tumor somewhere in the body, or for the treatment thereof. The amount of an AC133 expression product in PBMCs provides adequate information about different aspects and/or processes of an individual's body. Therefore, in a preferred embodiment, a method of the invention is provided wherein the sample comprises a peripheral blood mononuclear cell.

With a method of the invention, it is possible to determine whether an individual suffers from, or is at risk of suffering from, a disease. Moreover, it is possible to monitor therapy. Preferably, treatment of a tumor-related disease and/or a blood vessel-related disease is monitored with a method of the invention.

If a disease involves the presence of a tumor and/or an elevated level of angiogenesis, treatment typically comprises counteracting the tumor and/or the angiogenic process. Since such treatment is now easily monitored by a method of the invention, it is likewise easy to determine whether the treatment is effective.

In the art, many drugs are known for anti-tumor and/or anti-angiogenic treatment such as the following drugs 2ME2, ABT 510, ABT 751, angiostatin, ANGIOZYME™ anti-cancer agent, anti-VEGF RhuMAb, Apra (CT-2584), AVICINE™ anti-cancer agent, Benefin, BMS 275291, carboxyamidotriazole, cisplatin, CC 4047, CC 5013, CC 7085, CDC 801, CGP-41251 (PKC 412), CM 101, combretastatin A-4 Prodrug, DMXAA, EMD 121974, endostatin, enzastaurin HCI, flavopiridol, gemcitibine, Genistein (GCP), green tea extract, IM-862, ImmTher™ anti-cancer agent, interferon alpha, interleukin-12, IRESSA™ (ZD 1839), LY317615, Marimastat, METASTAT anti-cancer agent (Col-3), Neovastat agent, octreotide, Paclitaxel, penicillamine, PHOTOFRIN™ anti-cancer agent, PHOTOPOINT™, PI-88, PRINOMASTAT™ (AG-3340), PTK 787 (ZK 22584), RO 317453, Solimastat, squalamine, SU 101, SU11248, SU 5416, SU-6668, suradista (FCE 26644), suramin (METARET™), TETRATHIOMOLYBDATE™ anti-cancer agent, thalidomide, TNP-470, VEGF trap, ZD 6126, and/or VITAXIN® anti-cancer agent. Thus, in certain embodiments a method of the invention is provided wherein the treatment comprises the use of at least one of these drugs. Other drugs can be used during the treatment.

Now that a method is provided for the diagnosis of disease and/or monitoring of treatment of a disease, candidate compounds or methods can be tested for beneficial activity and/or possible side effects. Additionally, it can be tested whether compounds or methods are involved with causing/enhancing disease. Similar methods of the invention are performed for this purpose: after administration of such candidate compound to an individual, the amount of AC133 expression product in a sample from the individual is determined. If the amount of AC133 expression product is less indicative for disease as compared to the amount of AC133 expression product present in a sample of the individual prior to administration of the candidate compound, it indicates beneficial activity of the candidate compound. If, however, the amount of AC133 expression product is more indicative for disease as compared to the amount of AC133 expression product present in a sample of the individual prior to administration of the candidate compound, possible side effects (which may comprise involvement in causing/enhancing disease) of the candidate compound is indicated.

Thus provided is a method for determining therapeutic activity and/or possible side effects of a candidate compound comprising determining whether a sample from an individual, the individual having been provided with the candidate compound, comprises an expression product of AC133 in an amount that is indicative for disease or treatment thereof. A method of the invention is also suitable for (selective) toxin testing. The toxic activity of a candidate compound can be determined with a method of the invention. This way, the usefulness of such candidate compound for causing malfunctioning of a cellular organism, for instance by having a cytostatic or cytotoxic effect, can be determined.

In one aspect, therapeutic activity, possible side effects and/or toxic activity of a candidate compound is determined by administering the candidate compound to an essentially related organism, such as belonging to the same species or genus, and determining the amount of AC133 expression product in a sample from the essentially related organism. If the amount of AC133 expression product is indicative for disease or the treatment thereof, this also indicates toxic activity, side effects or therapeutic activity involved with the candidate compound in an essentially related organism. Therefore, for determining therapeutic activity, side effects and/or toxic activity of a candidate compound, it is not necessary to use exactly the same kind of organism in a method of the invention. An essentially related organism can also be used.

In one aspect, provided is a method for typing a sample of an individual suffering from, or at risk of suffering from, a disease comprising obtaining a sample from the individual and determining whether the sample comprises an expression product of AC133 in an amount indicative for the disease or for the treatment thereof.

Also provided is a method for monitoring treatment of an individual suffering from a disease comprising obtaining a sample from the individual, and determining whether the sample comprises an expression product of AC133 in an amount indicative for treatment.

In a preferred embodiment, a method is performed with at least one primer and/or probe as depicted in Table 2, or a functional part or derivative thereof. If at least one of the primers and/or probes is used, the sensitivity and reliability of a method of the invention is further improved.

In yet another aspect, provided is a use of a nucleic acid molecule comprising at least part of a sequence of AC133, or of an analogue of the nucleic acid molecule, for monitoring a treatment of an individual suffering from a disease. The presence of AC133 RNA in a sample of an individual can be detected by determining whether an AC133 nucleic acid or analogue thereof is capable of specifically hybridizing with RNA in a sample of the individual, preferably after amplification of the RNA of the sample. As defined herein, an AC133 nucleic acid or analogue thereof means a nucleic acid molecule comprising at least part of a sequence of AC133, or an analogue of the nucleic acid molecule. If hybridization takes place, it is indicative for the presence of AC133 RNA in the individual.

The (relative) amount of AC133 RNA can be determined using known methods in the art as described above. Hence, a nucleic acid molecule comprising at least part of a sequence of AC133 can be used in a method of the invention involving determining the amount of AC133 RNA in a sample. If one sample is obtained after the start of treatment or, preferably, if several samples are obtained after the start of treatment at different time points, the nucleic acid molecule comprising at least part of a sequence of AC133 or the analogue thereof is used for monitoring the treatment. A coding strand of DNA/RNA is capable of hybridizing with the complementary strand of a corresponding double-stranded nucleic acid sequence. Hence, a complementary strand of a certain coding strand is particularly suitable for detection of expression of the coding strand.

A part of a nucleic acid sequence of AC133 is defined herein as an AC133 nucleic acid sequence comprising at least 20 nucleotides, preferably at least 30 nucleotides, more preferably at least 50 nucleotides. A part and/or an analogue of an AC133 expression product is defined herein as a part and/or analogue that can be detected using essentially the same kind of detection method capable of detecting the expression product, although the sensibility of detection may differ. An analogue of an AC133 RNA or DNA molecule is defined herein as an RNA or DNA sequence that is at least 50% homologous to an AC133 RNA or DNA molecule. Preferably, the analogue is at least 60%, more preferably at least 70%, even more preferably at least 75%, yet more preferably at least 80%, even more preferably at least 85%, even yet more preferably at least 90%, even more preferably at least 95%, and most preferably at least 98% homologous to an AC133 RNA or DNA molecule, comprising at least part of a sequence of AC133.

In certain embodiments, the analogue of an AC133 RNA or DNA molecule has essentially the same properties as an AC133 RNA or DNA molecule in kind, albeit not necessarily in amount. A nucleotide mutation, replacement, alteration, addition and/or deletion may have taken place naturally and/or may have been introduced artificially, without essentially altering the detection capability of the analogue as compared to the detection of the AC133 RNA or DNA sequence. One may determine whether a given RNA or DNA sequence is an analogue of an AC133 RNA or DNA sequence, using techniques known in the art.

The invention also provides a diagnostic kit comprising at least one means for performing a method according to the invention, the kit comprising a nucleic acid molecule comprising at least part of a sequence of AC133, the part comprising at least 20 nucleotides. In certain embodiments, the part comprises at least 30 nucleotides. In another embodiment, the part comprises at least 50 nucleotides. Preferably, the kit further comprises suitable means for performing a nucleic acid amplification reaction.

Amplification of AC133 mRNA is preferred because amplification of AC133 mRNA enables detection of small initial amounts of AC133 mRNA in a sample. Moreover, an amount of mRNA more accurately reflects the actual status of AC133 expression because of its short half-life. In a preferred embodiment, the nucleic acid amplification reaction comprises NASBA, PCR, RT-PCR, TMA, bDNA, SDA or Rolling Circle amplification.

In certain embodiments, the nucleic acid amplification reaction comprises NASBA. Suitable primers and probes for amplifying and/or detecting AC133 RNA are listed in Table 2. These primers and probes are capable of amplifying and/or detecting both AC133-1 and AC133-2 isoforms. In one aspect, provided is a diagnostic kit that comprises at least one primer and/or probe as depicted in Table 2 or an analogue of the primer and/or probe.

A diagnostic kit described herein is particularly useful for carrying out a method of the invention. In yet another aspect, the invention therefore provides a use of a diagnostic kit of the invention for typing a sample of an individual suffering from, or at risk of suffering from, a disease. A use of a diagnostic kit of the invention for monitoring treatment of an individual suffering from a disease is also herewith provided.

Further provided is a primer and/or probe comprising a nucleic acid sequence as depicted in Table 2, or a functional part or analogue thereof. The primer and/or probe is particularly useful for performing a method of the invention.

### BRIEF DESCRIPTION OF THE DRAWINGS

FIG. 1: Amount of AC133 expression per 10,000 cells (quantified by Beta-Actin) in five patients treated with rhAngiostatin. The sample at day 1 (before therapy) was set at 100%.

FIG. 2: Number of circulating endothelial cells. The total amount of circulating endothelial cells was quantified by immunomagnetic separation with endothelium-specific antibody directed to CD146. The sample at day 1 (before therapy) was set at 100%.

FIGS. 3 through 5: Amount of AC133 expression per 10,000 cells (quantified by U1A) in patients treated with PrimMed01 and gemcitabine and cisplatin. The first sample (before therapy) was set at 100%. Only the sample before each course and the latest available sample during that course were plotted. FIG. 3: pre-treatment; FIG. 4: first course; FIG. 5: second course.

FIGS. 6 through 8: Amount of EST032 (SEQ ID NOS:7-9) expression per 10,000 cells (quantified by U1A) in patients treated with PrimMed01 and gemcitabine and cisplatin. The first sample (before therapy) was set at 100%. Only the sample before each course and the latest available sample during that course were plotted. FIG. 6: pre-treatment; FIG. 7: first course; FIG. 8: second course.

FIGS. 9 through 11: Amount of circulating endothelial cells (CEC) per ml. of blood in patients treated with PrimMed01 and gemcitabine and cisplatin as determined by FACS analysis with CD34. The first sample (before therapy) was set at 100%. Only the sample before each course and the latest available sample during that course were plotted. FIG. 9: pre-treatment; FIG. 10: first course; FIG. 11: second course.

FIGS. 12 and 13: Amount of mRNA expression per 10,000 cells (quantified by U1A) in patients treated with PrimMed01 and gemcitabine and cisplatin. The three samples represent the sample before pre-treatment, the sample just before course 1, and the sample just before course 2. The second sample (taken just before course 1) was set at 100%. FIG. 12: AC133 expression; FIG. 13: EST032 expression.

FIG. 14: AC133 and EST032 expression per 10,000 cells (quantified by U1A) in progressive cancer patients and healthy donors. Left panel: AC133; right panel: EST032. The average expression of AC133 and EST032 is significantly increased in patients as compared to healthy donors.

FIG. 15: AC133 and EST032 expression per 10,000 cells (quantified by U1A) in all samples, stratified on cancer growth. Left panel: AC133; right panel: EST032. A difference is considered to be significant if p value >0.05.

FIG. 16: Average AC133 expression per 10,000 cells (quantified by U1A) in all samples in two patients that were treated with GCSF.

FIG. 17: AC133 and EST032 expression per 10,000 cells (quantified by U1A) in all samples, stratified on cancer type. Left panel: AC133; right panel: EST032. A difference is considered to be significant if p value >0.05.

### DETAILED DESCRIPTION OF THE INVENTION

The invention is further explained in more detail by the following examples, which do not limit the invention in any way.

### EXAMPLES

### Example 1

### Patients and Samples

### Angiostatin Study

Five cancer patients (characteristics depicted in Table 1) who were not cured by treatment with other drugs were included in a phase I clinical trial of recombinant human angiostatin (rhAngiostatin). In this trial, designed to determine the toxicity of the drug, patients were treated with 7.5 mg/m²/day rhAngiostatin subcutaneously in a twice-daily schedule. Blood samples of the patients were taken at day 1 and day 28.

### Example 2

Peripheral blood mononuclear cells (PBMC) were isolated and approximately 1×10⁶ cells were dissolved in 1 ml L6 and stored at −80° C. 300 μl of the lysed-PBMC solution (containing approximately 300,000 PBMC) were added to a 1.5 ml Eppendorf tube containing 700 μl lysis buffer. The nucleic acid now present in the lysis buffer was further purified with the method described by Boom et al.¹ The isolated nucleic acid was eluted in 50 μl elution buffer. Usually, a dilution was made such that the equivalent of 10,000 cells/5 μl was used as input in NASBA amplification reactions.

In Table 2, the primers and probes used in these examples are summarized. Standard NASBA nucleic acid amplification reactions were performed in a 20 μl reaction volume and contained: 40 mM Tris-pH 8.5, 90 mM KCl, 12 mM MgCl₂, 5 mM dithiotreitol, 1 mM dNTPs (each), 2 mM rNTPs (each), 0.2 μM primer P1, 0.2 μM primer P2, 0.05 μM molecular beacon, 375 mM sorbitol, 0.1050 μg/ul bovine serum albumin, 6.4 units AMV RT, 32 units T7 RNA polymerase, 0.08 units RNase H and input nucleic acid. The complete mixture (except the enzymes) was, prior to adding the enzymes, heated to 65° C. in order to denature any secondary structure in the RNA and to allow the primers to anneal. (In the case of Beta-Actin, 2 units of MSP II were added. The mix was incubated at 37° C. for 15 minutes, followed by denaturation at 95° C.) After cooling the mixture to 41° C., the enzymes were added. The amplification took place at 41° C. for 90 minutes in a thermostatted fluorimeter (CytoFluor 2000 or EasyQ Reader) and the fluorescent signal of the molecular beacon probe was measured every 45 seconds.

To achieve quantification, a dilution series of target sequence was amplified and the time points at which the reactions became positive (the time to positivity, TTP) were plotted against the input amounts of nucleic acid. This way, a calibration curve was created that could be used to read TTP values of reactions with unknown amounts of input and deduce the input amount.

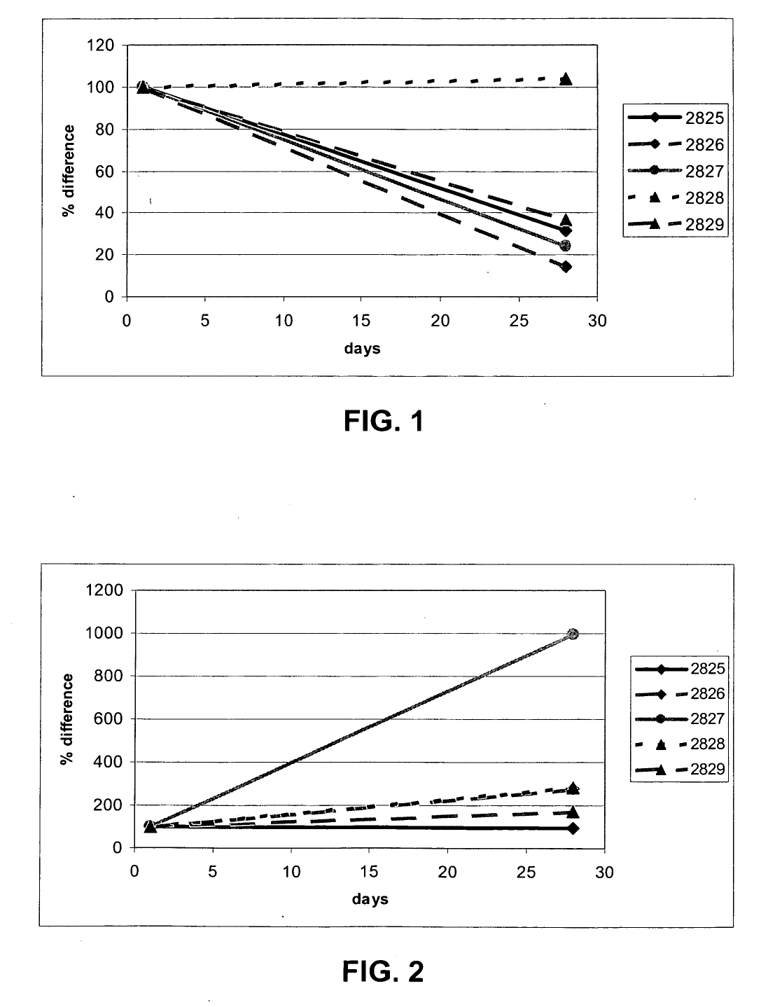

The AC133 expression per 10,000 cells was calculated after determination of the AC133 expression and the Beta-Actin copy number for each sample. The results are shown in FIG. 1. From this figure, it is clear that the AC133 expression per 10,000 cells in four of the five patients drops significantly. There seems to be no correlation with the kind of tumor, rate of progression (Table 1) or the total number of circulating endothelial cells (CECs; FIG. 2, quantified according to L. Beerepoot, Ann. Oncol. 15:139-145, 2004).

### Example 3

### Patients and Samples

### PrimMed01 Study

For this study, samples of 14 patients were available, but since we had no pre-treatment sample of two patients, they were not included in the analysis. The characteristics of the remaining 12 patients are depicted in Table 3. Patients received daily treatment with PrimMed01 (protein kinase inhibitor; anti-VEGF) for eight days. After this pre-treatment, the daily treatment with PrimMed01 was continued, but in addition, patients received a course of gemcitabine and cisplatin on day 15 (course 1) and day 36 (course 2).

Blood samples were taken before and after pre-treatment, before each course, and after 0, 2, 4, 8, and 24 hours after each course. After the first course, an extra sample was taken after 48 hours.

### Example 4

Isolation of nucleic acids from PBMC and NASBA amplification were performed as described in Example 2 with one modification: U1A was used instead of Beta Actin.

For the patients in the PrimMed01 study, not only the expression of AC133 per 10,000 cells, but also the expression of EST032 per 10,000 cells was determined.

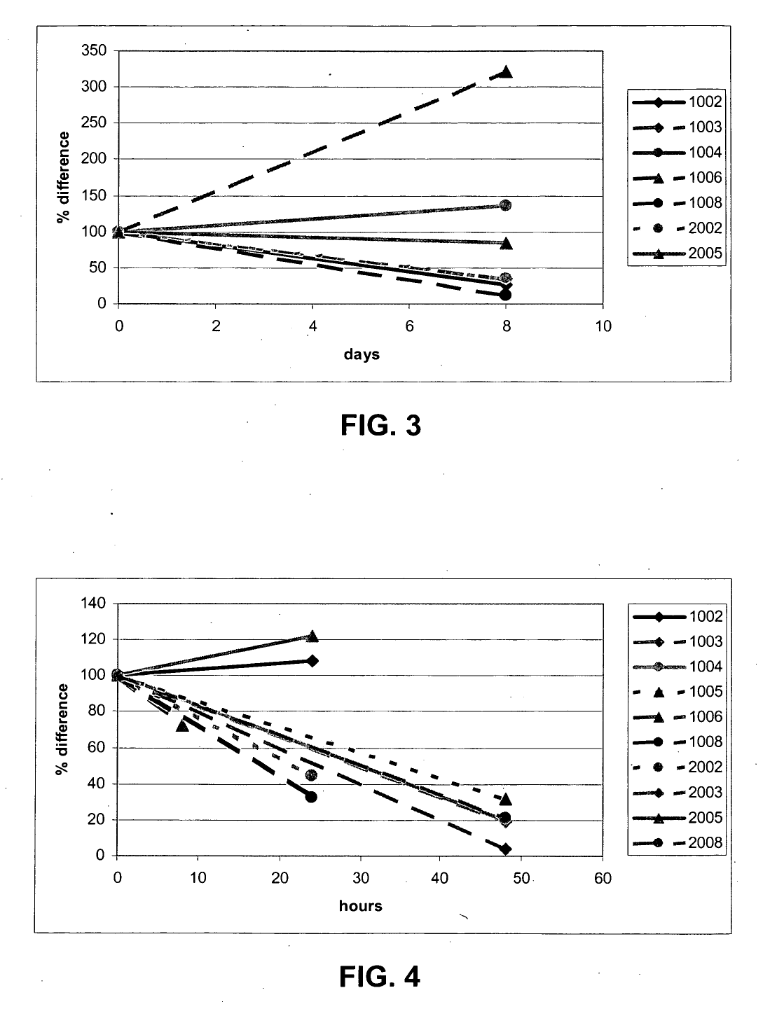

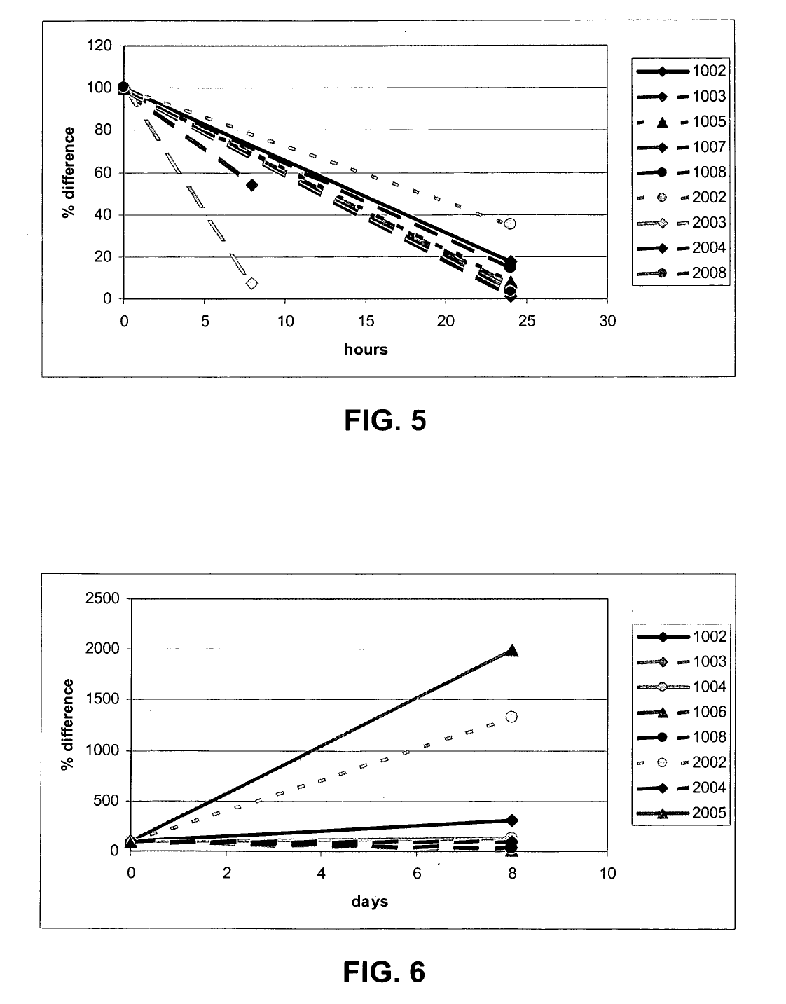

From FIGS. 3 through 5, it is clear that AC133 expression drops significantly during treatment with PrimMed01 and gemcitabine and cisplatin. During the pre-treatment, the AC133 expression does not seem to be affected, but here the observation period is much longer (eight days) compared to course 1 (follow up 48 hours) and course 2 (follow up 24 hours).

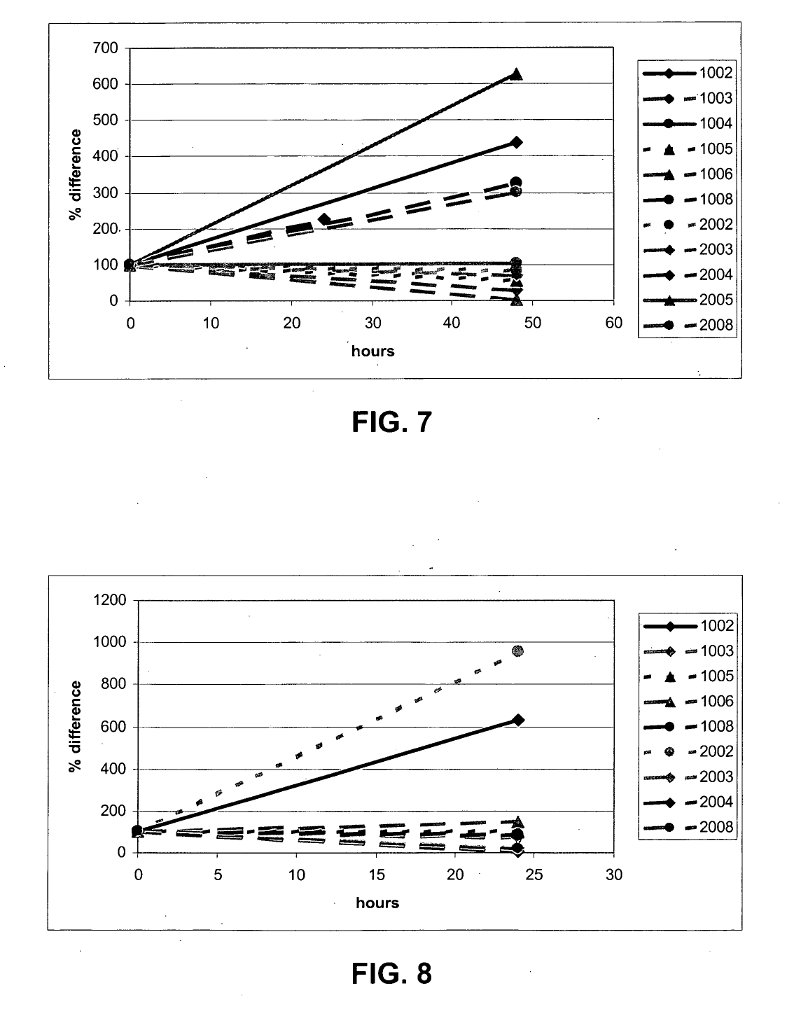

If we look at FIGS. 6 through 8, we clearly see that the expression of EST032 is not affected by therapy. It is, therefore, concluded that the decrease of AC133 expression is a specific effect of the therapy on the expression of AC133, and is not caused by the method.

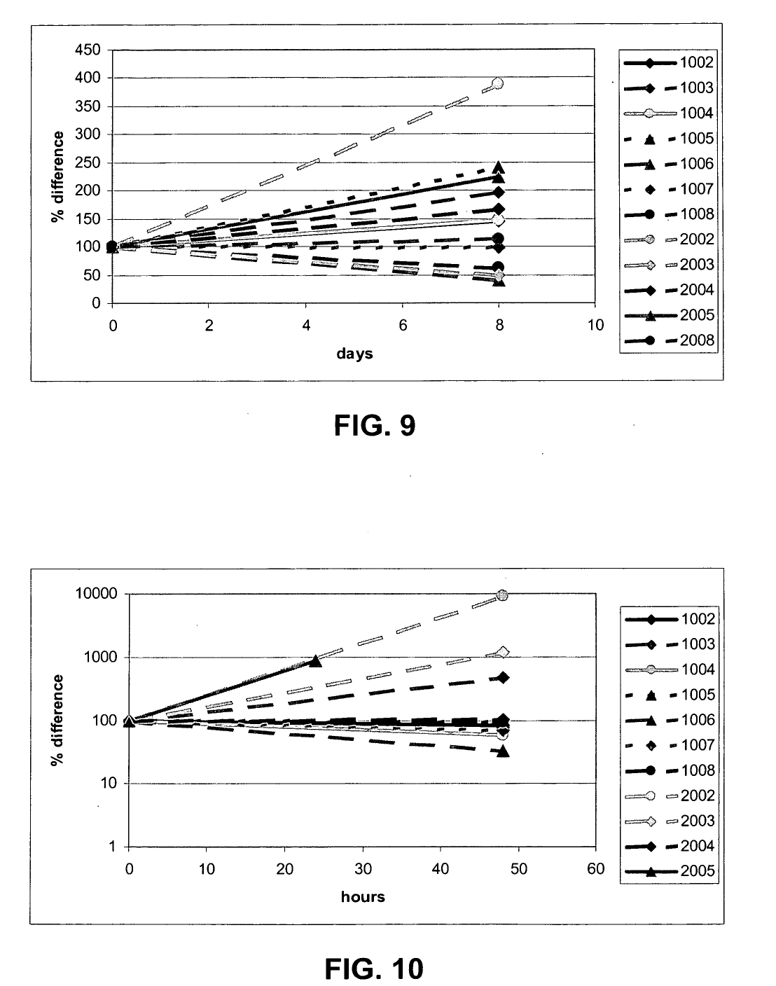

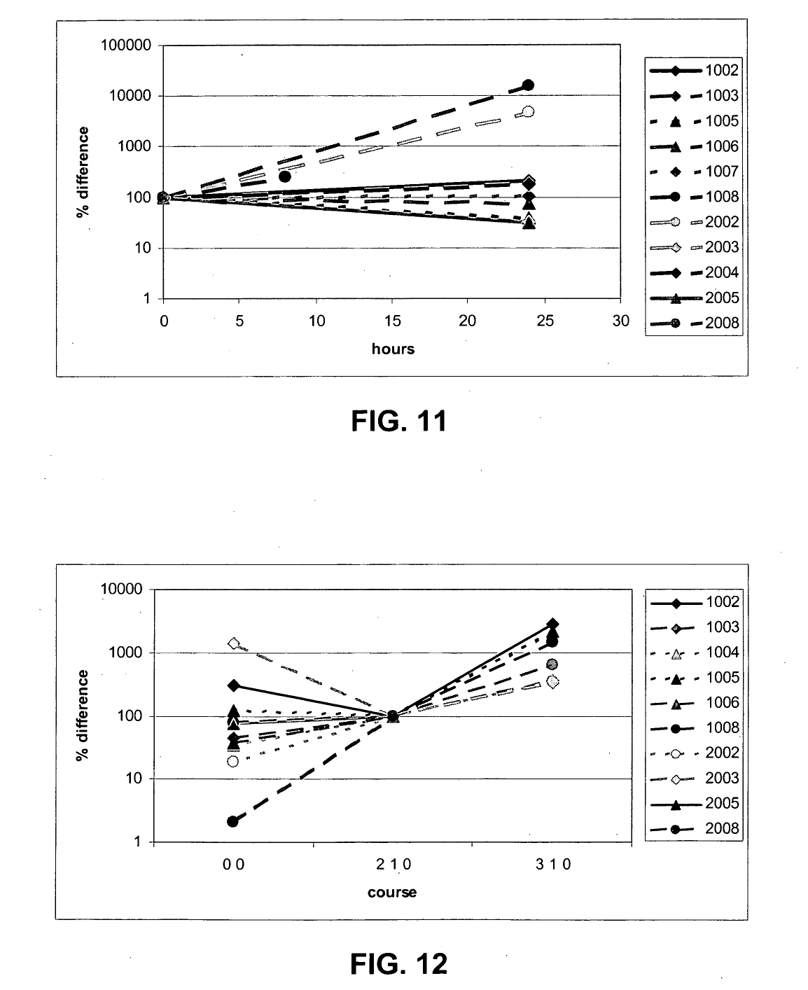

As is shown in FIGS. 9 through 11, there is also no association between AC133 expression and the amount of CECs.

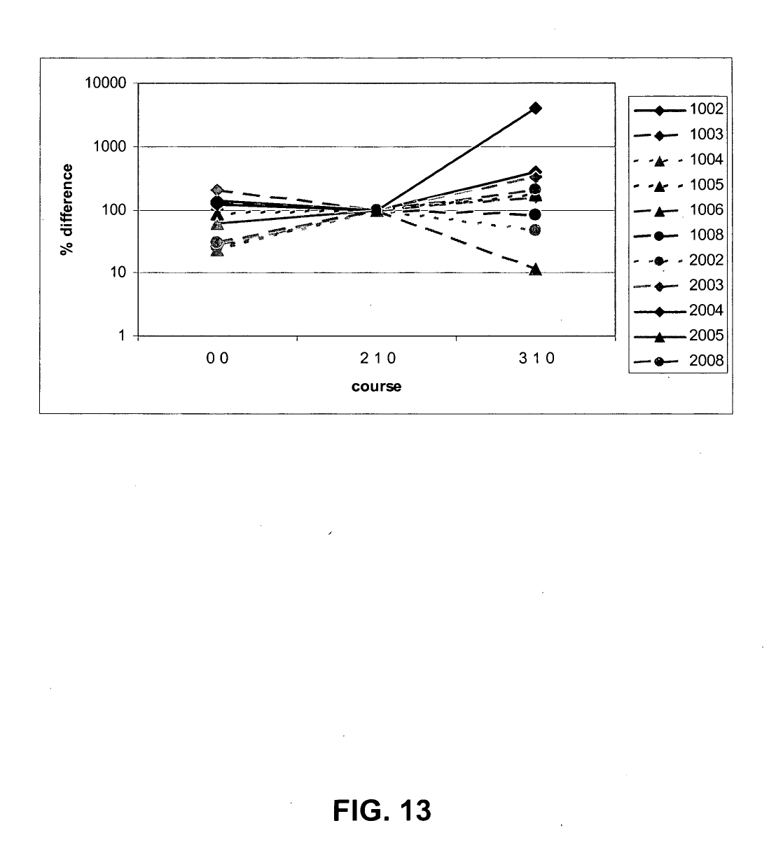

In FIGS. 12 and 13, we look at the effect of therapy over a longer period of time. In FIG. 12, the relative expression of AC133 just before each course is plotted (days 1, 15, and 35). After effective therapy (as determined in FIG. 4), an increase in AC133 expression is shown. That this effect on AC133 expression is a specific effect, and not caused due to the methods used, becomes clear if we look at the EST032 expression in the same samples (FIG. 13).

### Example 5

### Patients and Samples

### Healthy Donors Versus Untreated Patients

For this study, samples of 54 individuals were available, of which eight samples were from healthy donors and 46 were from patients who were not receiving any anti-cancer treatment at the moment of blood sampling.

### Example 6

Isolation of nucleic acids from PBMC and NASBA amplification were performed as described in Example 4.

In Table 4, we see that in cancer patients the expression of AC133 per 10,000 cells is almost one log higher compared to healthy donors. The difference in expression of EST032 between these two groups is much smaller.

### Example 7

The experiment of Example 6 was repeated with a higher amount of samples. For this study, samples of 174 individuals were available, of which 29 samples were from healthy donors and 145 were from patients who were not receiving any anti-cancer treatment at the moment of blood sampling.

Isolation of nucleic acids from PBMC and NASBA amplification were performed as described in Example 4.

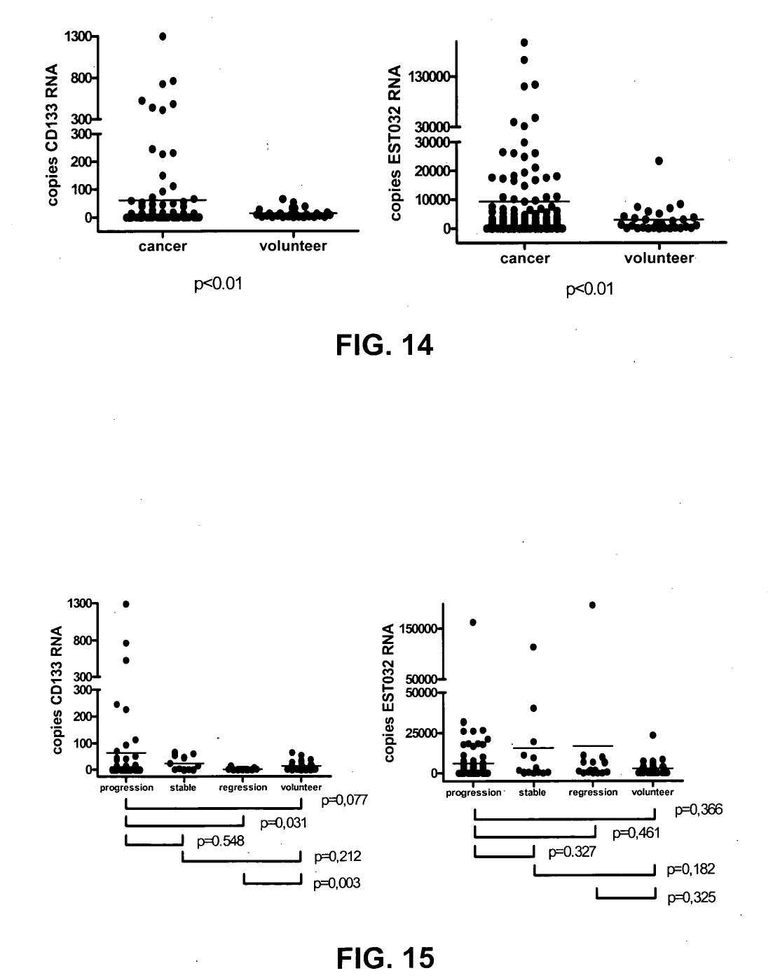

In FIG. 14, we see that in cancer patients the expression of AC133 per 10,000 cells is higher compared to healthy donors. The same holds true for difference in expression of EST032.

### Example 8

The patients from the previous examples differ, for example, in the way their cancer progresses. If all samples are divided in groups according to cancer growth (progressive disease, stable disease, regression, and healthy volunteer; FIG. 15), no differences in EST032 expression are found between the groups. However, if AC133 mRNA expression is compared, significant differences are found between patients with progression or regression, and volunteers and patients with regression.

### Example 9

Another way in which the samples from the previous examples can be analyzed is by dividing patients according to disease type. As shown in FIG. 17, especially patients with renal cell carcinoma (RCC) have significantly increased expression of EST032 and AC133.

### Example 10

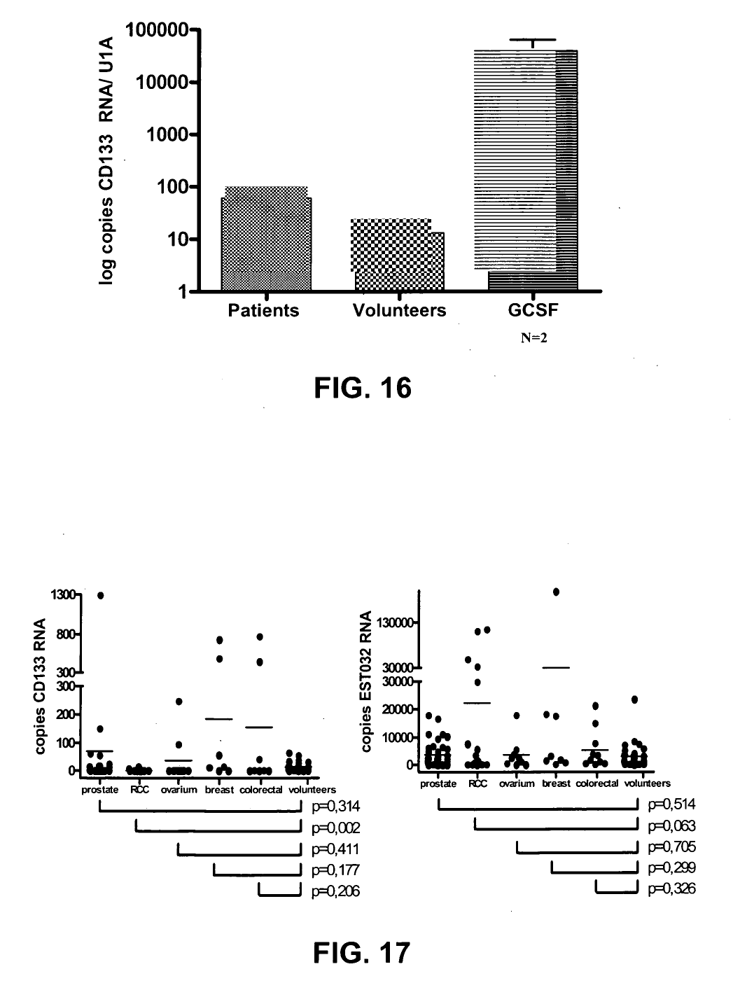

Two samples of patients who have been subjected to treatment with GCSF were tested. GCSF mobilizes stem cells from the bone marrow, so it is expected to find more AC133-expressing cells in the blood after a treatment with GCSF. It is clear from FIG. 16 that the AC133 expression in these two patients is much higher compared to volunteers and all the other patients.

TABLE 1
Characteristics of patients Angiostatin study

| patient | | | |
|---|---|---|---|
| number | age | tumor type | progression rate |
| 2825 | 41 | mouth bottom carcinoma | progressive |
| 2826 | 49 | adenoidcystic carcinoma | stable |
| 2827 | 49 | adenoidcystic carcinoma | stable |
| 2828 | 72 | renal cell carcinoma | stable |
| 2829 | 54 | colon carcinoma, liver/lung | progressive |
| | | metastases | |

TABLE 2
Sequences of primers and probes used

| | | |
|---|---|---|
| Name | Sequence¹ | SEQ ID NO: |

| | | |
|---|---|---|
| U1A P1 | 5′ AAT TCT AAT ACG ACT CAC | 1 |
| | TAT AGG GAG AGG CCC GGC | |
| | ATG TGG TGC ATA A 3′ | |
| U1A P2 | 5′ TGC GCC TCT TTC TGG GTG | 2 |
| | TT 3′ | |
| U1A MB | 5′ CGC ATG CTG TAA CCA CGC | 3 |
| | ACT CTC CTC GCA TGC G 3′ | |
| AC133 P1 | 5′ AAT TCT AAT ACG ACT CAC | 4 |
| | TAT AGG GAA GAA CAG GGA | |
| | TGA TGT TGG GTC TCA 3′ | |
| AC133 P2 | 5′ TTT CAA GGA CTT GCG AAC | 5 |
| | TCT CTT GA 3′ | |
| AC133 MB | 5′ CGA TCC AAG GAC AAG GCG | 6 |
| | TTC ACA GGA TCG 3′ | |
| EST032 P1 | 5′ AAT TCT AAT ACG ACT CAC | 7 |
| | TAT AGG GAG TAG CCC ACT | |
| | CAA GAG CTC TCT CCT GTT | |
| | GGT CCC T 3′ | |
| EST032 P2 | 5′ GCA TCT CTG TTC ATG ACT | 8 |
| | GTG TGA GCT CCT GTC CT 3′ | |
| EST032 MB | 5′ CGT ACG AAT GAC GTG CCC | 9 |
| | CTG CGA ATC GTA CG 3′ | |

1.
The T7 promoter part of primer P1 sequences is shown in italics, the stem sequences of the molecular beacon probes (MB) are shown in bold.
The molecular beacon probes were labeled at the 3′ end with DABCYL (the quencher) and at the 5′ end with a fluorescent label, which in the case of U1A is ROX, and in the case of AC133 and EST032 is 6-FAM.

TABLE 3
Characteristics of patients PrimMed01 study

| patient | | PrimMed01 | gemcitabine | cisplatin | | |
|---|---|---|---|---|---|---|
| number | gender | dose (mg) | dose (mg/m2) | dose (mg/m2) | tumor type | best response |
| 1002 | M | 350 | 1000 | 60 | esophagus | stable disease |
| 1003 | M | 350 | 1000 | 60 | mesothelioma | stable disease |
| 1004 | F | 350 | 1000 | 60 | pancreas | — |
| 1005 | F | 350 | 1250 | 60 | pancreas | stable disease |
| 1006 | M | 350 | 1250 | 60 | bladder | stable disease |
| 1007 | M | 350 | 1250 | 75 | pancreas | stable disease |
| 1008 | M | 500 | 1250 | 75 | ACUP | stable disease |
| 2002 | M | 350 | 1250 | 60 | prostate | partial regression |
| 2003 | M | 350 | 1250 | 75 | adenorenal | progressive disease |
| 2004 | M | 350 | 1250 | 75 | head and neck | partial response |
| 2005 | M | 350 | 1250 | 75 | melanoma | progressive disease |
| 2008 | M | 500 | 1250 | 75 | melanoma | partial response |

TABLE 4
RNA expression (expressed in log value)
in healthy donors vs. untreated patients

| | | donors | untreated patients |
|---|---|---|---|
| | number of samples | 8 | 46 |
| AC133 | average (per 10⁴ U1A) | 2.02 | 2.94 |
| EST032 | average (per 10⁴ U1A) | 3.29 | 3.97 |

### REFERENCES

1. Boom R., C. J. Sol, M. M. Salimans, C. L. Jansen, P. M. Wertheim-van Dillen and J. Van der Noordaa. Rapid and simple method for purification of nucleic acids. J. Clin. Microbiol. 28:495-503, 1990.
2. Van Gemen B., R. van Beuningen, A. Nabbe, D. Van Strijp, S. Jurriaans, P. Lens and T. Kievits. A one-tube quantitative HIV-1 RNA NASBA nucleic acid amplification assay using electrochemiluminescent- (ECL-) labeled probes. J. Virol. Methods 49:157-167, 1994.
3. Heid C. A., J. Stevens, K. J. Livak and P. M. Williams. Real-time quantitative PCR. Genome Res. 6:986-994, 1996.
4. Tyagi S. and F. R. Kramer. Molecular beacons: probes that fluoresce upon hybridization. Nat. Biotechnol. 14:303-308, 1996.
5. Leone G., H. van Schijndel, B. Van Gemen, F. R. Kramer and C. D. Schoen. Molecular beacon probes combined with amplification by NASBA enable homogeneous, real-time detection of RNA. Nucleic Acids Res. 26:2150-2155, 1998.
6. Piatak M., K. C. Luk, B. Williams and J. D. Lifson. Quantitative competitive polymerase chain reaction for accurate quantitation of HIV DNA and RNA species. Biotechniques 14:70-81, 1993.
7. De Baar M. P., M. W. van Dooren, E. de Rooij, M. Bakker, B. Van Gemen, J. Goudsmit and A. de Ronde. Single rapid real-time monitored isothermal RNA amplification assay for quantification of HIV-1 isolates from groups M, N, and O. J. Clin. Microbiol. 39(4):1378-1384, 2001.
8. Folkman J. Angiogenesis in cancer, vascular, rheumatoid and other disease. Nat. Med. 1:27-31, 1995.
9. Beerepoot L. V., N. Mehra, J. S. Vermaat, B. A. Zonnenberg, M. F. Gebbink and E. E. Voest. Increased levels of viable circulating endothelial cells are an indicator of progressive disease in cancer patients. Ann. Oncol. 15:139-145, 2004.
10. Miraglia S., W. Godfrey, A. H. Yin, K. Atkins, R. Warnke, J. T. Holden, R. A. Bray, E. K. Waller and D. W. Buck. A novel five-transmembrane hematopoietic stem cell antigen: isolation, characterization, and molecular cloning. Blood 90(12):5013-5021, 1997.
11. Yin A. H., S. Miraglia, E. D. Zanjani, G. Almeida-Porada, M. Ogawa, A. G. Leary, J. Olweus, J. Kearney and D. W. Buck. AC133, a novel marker for human hematopoietic stem and progenitor cells. Blood 90(12):5002-5012, 1997.
12. Yu Y., A. Flint, E. L. Dvorin and J. Bischoff. AC133-2, a novel isoform of human AC133 stem cell antigen. J. Biol. Chem. 277(23):20711-20716, 2002.
13. Mancuso P., A. Burlini, G. Pruneri, A. Goldhirsch, G. Martinelli and F. Bertolini. Resting and activated endothelial cells are increased in the peripheral blood of cancer patients. Blood 97(11):3658-3661, 2001.

1. A method for typing a sample of an individual suffering from, or at risk of suffering from, a disease, the method comprising:
determining whether the sample from the individual comprises an expression product of AC133 in an amount that is indicative of the disease or the treatment of the disease.

2. A method for monitoring treatment of an individual suffering from a disease, the method comprising:
determining whether a sample from the individual comprises an expression product of AC133 in an amount that is indicative of treatment of the disease.

3. The method according to claim 1, wherein the sample from the individual was obtained after initiation of the treatment.

4. The method according to claim 1, wherein the expression product comprises mRNA.

5. The method according to claim 1, 4 wherein the amount is quantified.

6. The method according to claim 1, further comprising:
comparing the amount with a reference value.

7. The method according to claim 1, further comprising:
comparing the amount of expression product with a first amount of expression product present in a sample that was obtained from the individual before the treatment.

8. The method according to claim 1, wherein the disease comprises a tumor.

9. The method according to claim 8, wherein the tumor is a progressive tumor.

10. The method according to claim 9, wherein the tumor is selected from the group consisting of mouth bottom carcinoma, adenoidcystic carcinoma, renal cell carcinoma, colon carcinoma, an esophagus tumor, mesothelioma, pancreatic tumor, bladder tumor, adenocarcinoma of unknown primary (ACUP), prostate tumor, renal adenocarcinoma, head cancer, neck cancer, malignant melanoma, and any combination thereof.

11. The method according to claim 9, wherein the tumor is selected from the group consisting of breast cancer, colorectal cancer, prostate cancer, ovarian cancer, and any combination thereof.

12. The method according to claim 1, wherein the disease is a blood vessel-related disease.

13. The method according to claim 12, wherein the disease is selected from the group consisting of heart disease, high blood pressure, transient ischemic attacks and strokes, psoriasis, Crohn's disease, rheumatoid arthritis, endometriosis, atherosclerosis, obesity, diabetes, diabetic retinopathy, macular degeneration, Alzheimer's disease, Peutz-Jegher's syndrome, multiple sclerosis, systemic lupus erythematosus, Wegener's granulomatosis, vasculitis, sickle cell disease, thalassemia, angina, and any combination thereof.

14. The method according to claim 1, wherein the sample comprises a significant amount of non-endothelial cells.

15. The method according to claim 1, wherein the sample is an essentially cell-free sample.

16. The method according to claim 1, wherein the sample comprises a blood sample.

17. The method according to claim 16, wherein the sample comprises a peripheral blood mononuclear cell.

18. The method according to claim 1, wherein the treatment comprises the use of at least one of the following drugs: 2ME2, ABT 510, ABT 751, angiostatin, ANGIOZYME™ anti-cancer agent, anti-VEGF RhuMAb, Apra (CT-2584), AVICINE™ anti-cancer agent, Benefin, BMS 275291, carboxyamidotriazole, cisplatin, CC 4047, CC 5013, CC 7085, CDC 801, CGP-41251 (PKC 412), CM 101, combretastatin A-4 Prodrug, DMXAA, EMD 121974, endostatin, enzastaurin HCI, flavopiridol, gemcitibine, Genistein (GCP), green tea extract, IM-862, ImmTher™ anti-cancer agent, interferon alpha, interleukin-12, IRESSA™ (ZD 1839), LY317615, Marimastat, METASTAT™ anti-cancer agent (Col-3), Neovastat agent, octreotide, Paclitaxel, penicillamine, PHOTOFRIN™ anti-cancer agent, PHOTOPOINT™, PI-88, PRINOMASTAT™ (AG-3340), PTK 787 (ZK 22584), RO 317453, Solimastat, squalamine, SU 101, SU11248, SU 5416, SU-6668, suradista (FCE 26644), suramin (METARET™), TETRATHIOMOLYBDATE™ anti-cancer agent, thalidomide, TNP-470, VEGF trap, ZD 6126, and/or VITAXIN® anti-cancer agent.

19. The method according to claim 1, wherein the sample is obtained within a month of initiation of the treatment.

20. The method according to claim 19, wherein the sample is obtained within a week of initiation of the treatment.

21. The method according to claim 20, wherein the sample is obtained within two days of initiation of the treatment.

22. The method according to claim 1, comprising interacting the sample with at least one primer and/or probe selected from the group consisting of SEQ ID Nos:1-9.

23. A method of monitoring an individual's treatment, the individual suffering from a disease, the method comprising:
analyzing a sample from the individual with a nucleic acid molecule comprising at least part of a sequence of AC133 or an analogue thereof.

24. The method according to claim 23, wherein the disease comprises a progressive tumor.

25. A method of determining whether a tumor is progressive, the method comprising:
analyzing a sample associated with the tumor with:
a nucleic acid molecule comprising at least part of a sequence of AC133,
an analogue of a nucleic acid molecule comprising at least part of a sequence of AC133, or
a molecule able to specifically bind an AC133 expression product for determining whether a tumor is progressive.

26. A kit comprising:
a nucleic acid molecule comprising at least part of a sequence of AC133.

27. The kit of claim 26, further comprising:
means for performing a nucleic acid amplification reaction.

28. The kit of claim 27, wherein the nucleic acid amplification reaction is selected from the group consisting of NASBA, PCR, RT-PCR, TMA, bDNA, SDA, and Rolling Circle amplification.

29. The kit of claim 26, wherein the kit comprises at least one primer and/or probe selected from the group consisting of SEQ ID Nos:1-9.

30. A method of monitoring treatment of a subject suffering from a disease, the method comprising:
obtaining a biological sample from the subject, and
analyzing the biological sample with the kit of claim 26.

31. The method according to claim 30, wherein the disease comprises a progressive tumor.

32. A method of monitoring typing a sample of an individual suffering from, or at risk of suffering from, a disease, the method comprising:
analyzing the sample with the kit of claim 26.

33. A primer and/or probe comprising a nucleic acid sequence selected from the group consisting of SEQ ID Nos: 1-9, and an analogue of any thereof.

34. A method for typing a sample of an individual suffering from, or at risk of suffering from, a disease, the method comprising:
obtaining a sample from the individual, and
determining whether the sample comprises an expression product of AC133 in an amount that is indicative for the disease or for the treatment of the disease.

35. A method for monitoring treatment of an individual suffering from a disease, the method comprising:
obtaining a sample from the individual, and
determining whether the sample comprises an expression product of AC133 in an amount that is indicative of the treatment.

36. The method according to claim 34, wherein the disease comprises the presence of a progressive tumor.
